(19) United States
(12) Patent Application Publication
(10) Pub. No.: US 2019/0298277 A1
(43) Pub. Date: Oct. 3, 2019

(54) SURGICAL POSITIONING APPARATUS, POSITIONING SYSTEM AND POSITIONING METHOD

(71) Applicant: TINAVI MEDICAL TECHNOLOGIES CO., LTD., Beijing (CN)

(72) Inventors: Songgen ZHANG, Beijing (CN); Yinyan LI, Beijing (CN); Jin XU, Beijing (CN)

(73) Assignee: TINAVI MEDICAL TECHNOLOGIES CO., LTD, Beijing (CN)

(21) Appl. No.: 16/307,476

(22) PCT Filed: Oct. 27, 2016
(86) PCT No.: PCT/CN2016/103502

§ 371 Date: Dec. 5, 2018

(30) Foreign Application Priority Data
Jun. 8, 2016 (CN) 201610403217.6

(51) Int. Cl. A61B 6/04; A61B 6/03; A61B 6/00; A61B 34/32; A61B 6/02

(52) U.S. Cl. CPC A61B 6/0492; A61B 6/035; A61B 6/4085; A61B 6/582; A61B 6/022; A61B 6/5247; A61B 34/32

(57) ABSTRACT

The disclosure relates to a surgical positioning apparatus, positioning system and positioning method. The positioning apparatus comprises a bracket, on which three or more reflecting balls for reflecting infrared and four or more positioning markers opaque to X-ray are provided. The disclosure provides both the reflecting balls for reflecting infrared and the positioning markers on the bracket, thus the reflecting balls can be identified by an optical tracking device, and the positioning markers can be scanned and identified by a three-dimensional device.

### CROSS REFERENCE

The present disclosure claims the benefit of an International Patent Application No. PCT/CN2016/103502 filed on Oct. 27, 2016, which claims the benefit of a Chinese Patent Application No. 201610403217.6 filed on Jun. 8, 2016. The above patent applications are incorporated entirely by reference in the disclosure.

### TECHNICAL FIELD

The disclosure relates to a surgical positioning apparatus, positioning system and positioning method, which belongs to the field of surgical positioning technology.

### BACKGROUND

As cross application of robotic technology and medical science develops rapidly, research on a variety of medical robots becomes a hot topic and medical robots are increasingly widely used in medical field. Surgical robot is one of the frontier research hotspots. Currently surgical robots have been widely used in the aspects of neurosurgery department, prosthetic replacement, urology department, gallbladder extirpation and so on. Surgical operation by a robot is typically advantageous in some aspects compared to manual operation by a doctor. For example, positioning of robots is more precise, and grabbing of robot arms is more stable and powerful, which can avoid fatigue resulting from long time surgery of a surgeon and improve precision, stability and safety. The robotic surgery can shorten surgery time, reduce damage to patients and doctors by X-ray during surgery process and protect the health of patients and doctors.

As the science and technology develop, intraoperative spiral CT machines begin to popularize in hospitals, and it has large imaging range and high definition compared to cone beam CT machines and other intraoperative three-dimensional image devices, and is particularly suitable to cooperate with robots to carry out high precision locating surgery. In robot navigation, it is necessary to establish interrelation among CT images, patients and robot positioning system. A CT recognition calibrator for image registration is generally held by a robot arm and placed near a scanning part of a patient. The CT recognition calibrator is configured to form a specific positioning marker distribution in a CT image. According to the positioning marker distribution, it is possible to achieve spatial positioning calculation and determine the relation among the robot, the CT image and the patient, thereby determining a surgical route. However, intraoperative spiral CT machines are generally operated with stationary patients and moving scanners. Since the scanner has larger volume and requires large space in moving, it is possible to result in the risk of collision if the robot enters the moving area. In terms of safe application, it is necessary to remove the independent CT recognition calibrator such that the robot arm can keep away from the patient and from potential collision area. However, if the CT recognition calibrator is removed, the planning of the robotic surgery route cannot be achieved.

### SUMMARY

With respect to the above problem, an object of the present disclosure is to provide a surgical positioning apparatus applicable to spiral CT machines with very small footprint and a positioning system and positioning method based on the same.

In order to achieve the above object, the disclosure provides a surgical positioning apparatus, characterized in that the surgical positioning apparatus comprises a bracket, on which three or more reflecting balls for reflecting infrared and four or more positioning markers opaque to X-ray are provided.

A distance between any two of the reflecting balls is greater than 50 mm and a difference between various distances between the reflecting balls is greater than 5 mm; and at least three of the reflecting balls are at an angle less than or equal to 75°.

The positioning markers are divided into two groups, wherein each group comprises three or more positioning markers, and the distribution of positioning markers in each group on the bracket satisfies the following conditions: a distance between any two positioning markers is greater than 20 mm and a difference between various distances between the positioning markers is greater than 5 mm; and at least three of the positioning markers are at an angle of less than or equal to 75°.

The disclosure further provides a surgical positioning system, characterized in that the surgical positioning system comprises a surgical robot, a host computer, an optical tracking device, a robot tracer, a three-dimensional imaging device and a surgical positioning apparatus; the surgical robot is a robot arm with at least three translational degrees of freedom and three rotational degrees of freedom; the host computer is electrically connected to the surgical robot for controlling the motion of the surgical robot; the robot tracer is mounted at a tip of the surgical robot; the surgical positioning apparatus is fixed on a patient's body; the three-dimensional imaging device is configured to scan the surgical positioning apparatus to form a three-dimensional image including positioning markers, and the host computer is configured to identify and match the positioning markers in the image and the positioning markers on the surgical positioning apparatus; and the optical tracking device is configured to track the robot tracer and the surgical positioning apparatus and transmit position data to the host computer.

The three-dimensional imaging device is a spiral CT machine or C-type or O-type cone beam CT machine.

The disclosure further provides a surgical positioning method comprising the steps of: (1) placing a surgical positioning apparatus fixed on a patient's body in a field of view of a three-dimensional imaging device to scan it; obtaining, with the three-dimensional imaging device, an image of positioning markers on the surgical positioning apparatus and transmitting the image to a host computer; and during the three-dimensionally scanning of the surgical positioning apparatus, obtaining, with an optical tracking device, coordinates of a robot tracer and the surgical positioning apparatus and transmitting the coordinates to the host computer; (2) repeatedly comparing, with the host computer, the positioning markers in the image to preset geometrical characteristics of the positioning markers, in order to identify and match the positioning markers in the surgical positioning apparatus and the positioning markers in the image; and (3) calculating, with the host computer, a transformation relation among the patient, the image and the surgical robot in a coordinate system in which the surgical positioning apparatus is located according to a rotation matrix and a translation vector between a coordinate vector of a robot tracer and a coordinate vector of the surgical positioning apparatus, and choosing one of a patient coordinate system, a robot coordinate system, a robot base coordinate system and an image coordinate system as the world coordinate system, and outputting a transformation relation by which the patient, the image and the surgical robot are brought into the common world coordinate system, as a result of image registration

In the step (2), the process of identifying the positioning markers on the surgical positioning apparatus and the positioning markers in the image comprises the following steps: (a) dividing the positioning markers on the surgical positioning apparatus into group I and group II, wherein each group comprises three or more positioning markers; (b) reading information on the positioning markers in group I and the group II in step (a) and information on the surgical positioning apparatus, and reading the image obtained by scanning in step (1); (c) performing threshold-segmentation on the image obtained in step (b) and extracting and generating valid polygon data; (d) fitting and deciding the polygon data obtained in step (c) according to the information on the surgical positioning apparatus obtained in step (b), thereby screening out the positioning markers in the image; (e) calculating a distance between every two positioning markers of the positioning markers in the image obtained in step (d); (f) choosing 3 positioning markers from the positioning markers on the surgical positioning apparatus in group I to constitute a triangle as a triangular template, and searching for a triangle in the image approximately identical to the triangular template; if there is no such triangle, choosing 3 positioning markers from the positioning markers on the surgical positioning apparatus in group II to constitute a triangle as a triangular template, and searching for a triangle in the image approximately identical to the triangular template; and if there is still no such triangle, choosing the positioning markers on the surgical positioning apparatus from group I and group II to constitute a triangle as a triangular template, searching for a triangle in the image approximately identical to the triangular template; and (g) matching serial numbers of respective vertices of the paired congruent triangles according to a one-to-one correspondence, to form a matching vertex pair, and searching for an image positioning marker outside of the triangular template in the image corresponding to a positioning marker on the surgical positioning apparatus with reference to the congruent triangular template, until all image positioning markers match the positioning markers on the surgical positioning apparatus.

The disclosure provides the above technical solutions and thus possesses the following advantages. First, the disclosure provides both the reflecting balls for reflecting infrared and the positioning markers on the bracket, thus the reflecting balls can be identified by an optical tracking device, and the positioning markers can be scanned and identified by a three-dimensional device. This provides a technical basis for image registration. It is more important that the disclosure has high integration level and small footprint, and is applicable to a positioning system using a spiral CT as imaging device. Second, the positioning method according to the disclosure can be implemented by programs. The positioning method can achieve intraoperative automatic registration without manual intervention and has good registration precision. Third, the method of the disclosure has high positioning precision, which provides a good basis for surgical route planning.

### BRIEF DESCRIPTION OF THE DRAWINGS

The disclosure will hereafter be described with reference to the accompanying drawings. It should be understood, however, that the accompanying drawings provide a better understanding of the present disclosure and are not meant to limit the scope of the disclosure.

FIG. 1 is a schematic structural view of a surgical positioning apparatus according to the disclosure.

FIG. 2 is a schematic structural view of a surgical positioning system according to the disclosure.

### DETAILED DESCRIPTION

The disclosure will be described in detail below in combination with the drawings and embodiments.

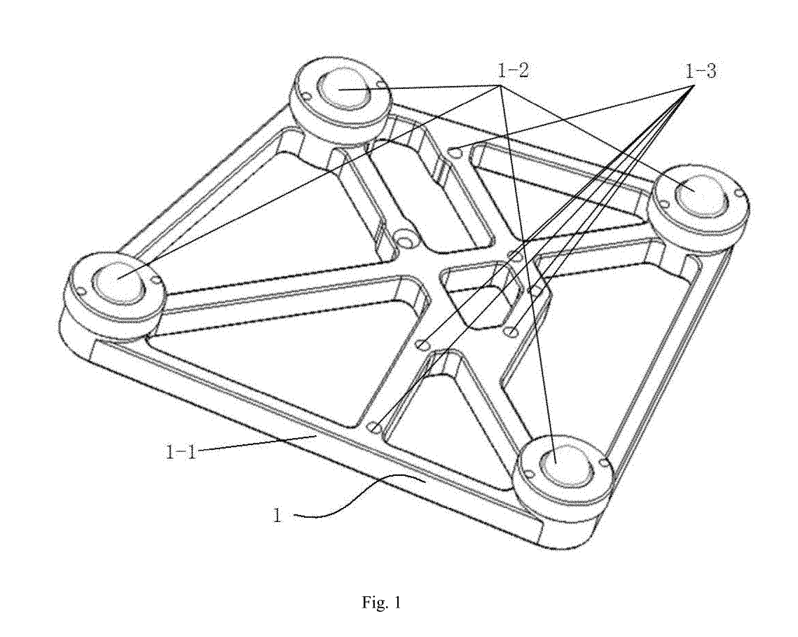

As shown in FIG. 1, the disclosure provides a surgical positioning apparatus 1, which comprises a bracket 1-1. Three or more reflecting balls 1-2 for reflecting infrared are provided on the bracket 1-1. The reflecting balls 1-2 serve as optical tracking markers which can be identified by an optical tracking device, such that optical tracking of the surgical positioning apparatus 1 can be achieved. Four or more positioning markers 1-3 opaque to X-ray are further provided on the bracket 1-1. The positioning markers 1-3 can be identified in a three-dimensional image formed through scanning by a three-dimensional imaging device such as CT machine, such that a host computer can carry out spatial positioning calculation according to the distribution of the positioning markers 1-3, thereby planning a surgical route.

In the above embodiment, the distribution of the reflecting balls 1-2 on the bracket 1-1 satisfies the following conditions: (1) a distance between any two of the reflecting balls 1-2 is greater than 50 mm, and a difference between various distances between the reflecting balls is greater than 5 mm (taking three reflecting balls A, B and C as an example, there are three line segments AB, AC and BC, and then the lengths of said three line segments are greater than 50 mm, 55 mm and 60 mm in turn); and (2) at least three of the reflecting balls 1-2 are at an angle less than or equal to 75°.

In the above embodiment, the positioning markers 1-3 are divided into two groups, wherein each group includes three or more positioning markers 1-3. The same positioning marker 1-3 may be repeatedly assigned to multiple groups. The distribution of the positioning markers 1-3 in each group satisfies the following conditions (the disclosure is not limited to this type of group dividing): (1) a distance between any two of the positioning markers 1-3 is greater than 20 mm, and a difference between various distances between the positioning markers is greater than 5 mm (taking three positioning markers A, B and C as an example, there are three line segments AB, AC and BC, and then the lengths of said three line segments are greater than 20 mm, 25 mm and 30 mm in turn); and (2) at least three of the positioning markers 1-3 are at an angle less than or equal to 75°.

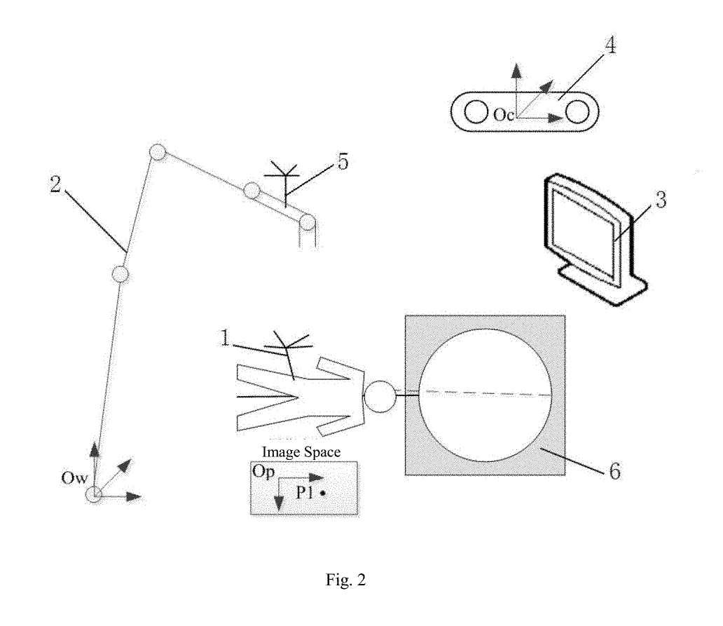

As shown in FIG. 2, the disclosure also provides a surgical positioning system, which comprises a surgical positioning apparatus 1, a surgical robot 2, a host computer 3, an optical tracking device 4, a robot tracer 5 and a three-dimensional imaging device 6. The surgical robot 2 is a robot arm with at least three translational degrees of freedom and three rotational degrees of freedom. The host computer 3 is electrically connected to the surgical robot 2 for controlling the motion of the surgical robot 2. The robot tracer 5 is mounted at a tip of the surgical robot 2. The surgical positioning apparatus 1 is fixed on a patient's body. The three-dimensional imaging device 6 is configured to scan the surgical positioning apparatus 1 to form a three-dimensional image including the positioning markers 1-3. The host computer 3 is configured to identify and match the positioning markers in the image and the positioning markers 1-3 on the surgical positioning apparatus 1. The optical tracking device 4 is configured to track the robot tracer 5 and the surgical positioning apparatus 1 and transmit position data to the host computer 3.

In the above embodiment, the three-dimensional imaging device 6 can be a spiral CT machine or can also be an O-type cone beam CT machine (O-Arm).

The positioning method implemented based on the above surgical positioning system of the disclosure comprises the following steps.

Step (1) comprises: fixing the surgical positioning apparatus 1 to a patient and placing the surgical positioning apparatus 1 in a field of view of the three-dimensional imaging device 6 to scan it; obtaining, with the three-dimensional imaging device 6, an image of the positioning markers 1-3 on the surgical positioning apparatus 1 and transmitting the image to the host computer 3; during the three-dimensionally scanning of the surgical positioning apparatus 1, obtaining, with the optical tracking device 4, coordinates of the robot tracer 5 and the surgical positioning apparatus 1 and transmitting the coordinates to the host computer 3.

Step (2) comprises: repeatedly comparing, with the host computer 3, the positioning markers in the image to preset geometrical characteristics of the positioning markers, in order to identify and match the positioning markers 1-3 in the surgical positioning apparatus 1 and the positioning markers in the image.

Step (3) comprises: calculating, with the host computer 3, a transformation relation among the patient, the image and the surgical robot 2 in a coordinate system in which the surgical positioning apparatus 1 is located according to a rotation matrix and a translation vector between a coordinate vector of a robot tracer 5 and a coordinate vector of the surgical positioning apparatus 1; choosing one of a patient coordinate system, a robot coordinate system, a robot base coordinate system and an image coordinate system as the world coordinate system; and outputting a transformation relation by which the patient, the image and the surgical robot 2 are brought into the common world coordinate system, as a result of image registration.

In the registered image, a doctor draws a surgical route as needed in treatment, determines a needle entering point (or a needle exiting point) P according to the surgical route, and calculates a world coordinate of the point P in the world coordinate system. After the world coordinates of the needling entering point and the needle exiting point are determined respectively, the spatial coordinates of the surgical route are expressed as a straight line in the world coordinate system, which is output as surgical planning. After the surgical route is calculated, it is possible to control the surgical robot 2 to move precisely, directing a guiding device (a tubular needle holder for fixing the needling entering route) connected to the tip of the surgical robot 2 to the surgical route. During the above process, the optical tracking device 4 with real-time tracking function monitors the surgical positioning apparatus 1 (i.e. the movement of the patient) in real time, and calculates the direction and magnitude of the movement. The surgical robot 2 can correct its own motion according to the direction and magnitude of the movement, thereby ensuring precise consistency of the guiding device with the planned route.

In the step (2), the specific process of identifying the positioning markers 1-3 on the surgical positioning apparatus 1 and the positioning markers in the image comprises the following substeps.

Substep (a) comprises: dividing the positioning markers 1-3 on the surgical positioning apparatus 1 into group I and group II, wherein each group includes three or more positioning markers 1-3, and each positioning marker may be repeatedly assigned to different groups.

Substep (b) comprises: reading information on the positioning markers in group I and the group II in step (a) and information on the surgical positioning apparatus 1, and reading the image obtained by scanning in step (1).

Substep (c) comprises: performing threshold-segmentation on the image obtained in step (b) and extracting and generating valid polygon data.

Substep (d) comprises: fitting and deciding the polygon data obtained in step (c) according to the information on the surgical positioning apparatus 1 obtained in step (b), thereby screening out the positioning markers in the image.

Substep (e) comprises: calculating a distance between every two positioning markers of the positioning markers in the image obtained in step (d).

Substep (f) comprises: choosing 3 positioning markers from the positioning markers on the surgical positioning apparatus in group I to constitute a triangle as a triangular template, and searching for a triangle in the image approximately identical to the triangular template; if there is no such triangle, choosing 3 positioning markers from the positioning markers on the surgical positioning apparatus in group II to constitute a triangle as a triangular template, and searching for a triangle in the image approximately identical to the triangular template; and if there is still no such triangle, choosing the positioning markers on the surgical positioning apparatus from group I and group II to constitute a triangle as a triangular template, searching for a triangle in the image approximately identical to the triangular template.

Substep (g) comprises: matching serial numbers of respective vertices of the paired congruent triangles according to a one-to-one correspondence, to form a matching vertex pair, and searching for an image positioning marker outside of the triangular template in the image corresponding to a positioning marker on the surgical positioning apparatus with reference to the congruent triangular template, until all image positioning markers match the positioning markers on the surgical positioning apparatus.

The disclosure is only described through the above embodiments, and the structures, set positions and connection of various parts may be changed. On the basis of the technical solution of the disclosure, all the improvements or equivalent variations of individual parts according to the principle of the disclosure shall not be excluded from the protection scope of the disclosure.

1. (canceled)

2. The surgical positioning apparatus of claim 3, wherein a distance between any two of the reflecting balls is greater than 50 mm and a difference between various distances between the reflecting balls is greater than 5 mm; and at least three of the reflecting balls are at an angle less than or equal to 75°.

3. A surgical positioning apparatus, comprising a bracket, on which three or more reflecting balls for reflecting infrared and four or more positioning markers opaque to X-ray are provided,
wherein the positioning markers are divided into two groups, wherein each group comprises three or more positioning markers, and the distribution of positioning markers in each group on the bracket satisfies the following conditions: a distance between any two positioning markers is greater than 20 mm and a difference between various distances between the positioning markers is greater than 5 mm; and at least three of the positioning markers are at an angle of less than or equal to 75°.

4. A surgical positioning system, comprising a surgical robot, a host computer, an optical tracking device, a robot tracer, a three-dimensional imaging device and a surgical positioning apparatus;
the host computer is electrically connected to the surgical robot for controlling the motion of the surgical robot;
the surgical positioning apparatus comprises a bracket, on which three or more reflecting balls for reflecting infrared and four or more positioning markers opaque to X-ray are provided;
the robot tracer is mounted at a tip of the surgical robot;
the surgical positioning apparatus is configured to be fixed on a patient's body;
the three-dimensional imaging device is configured to scan the surgical positioning apparatus to form a three-dimensional image including positioning markers, and the host computer is configured to identify and match the positioning markers in the image and the positioning markers on the surgical positioning apparatus; and
the optical tracking device is configured to track the robot tracer and the surgical positioning apparatus and transmit position data to the host computer.

5. The surgical positioning system of claim 4, wherein the three-dimensional imaging device is a spiral CT machine or C-type or O-type cone beam CT machine.

6. A surgical positioning method, comprising the steps of:
(1) placing a surgical positioning apparatus fixed on a patient's body in a field of view of a three-dimensional imaging device to scan it, wherein the surgical positioning apparatus comprises a bracket, on which three or more reflecting balls for reflecting infrared and four or more positioning markers opaque to X-ray are provided; obtaining, with the three-dimensional imaging device, an image of positioning markers on the surgical positioning apparatus and transmitting the image to a host computer; and during the three-dimensionally scanning of the surgical positioning apparatus, obtaining, with an optical tracking device, coordinates of a robot tracer and the surgical positioning apparatus and transmitting the coordinates to the host computer, wherein the robot tracer is mounted at a tip of a surgical robot;
(2) repeatedly comparing, with the host computer, the positioning markers in the image to preset geometrical characteristics of the positioning markers, in order to identify and match the positioning markers in the surgical positioning apparatus and the positioning markers in the image; and
(3) calculating, with the host computer, a transformation relation among the patient, the image and the surgical robot in a coordinate system in which the surgical positioning apparatus is located.

7. The surgical positioning method of claim 6, wherein in the step (2), the process of identifying the positioning markers on the surgical positioning apparatus and the positioning markers in the image comprises the following steps:
(a) dividing the positioning markers on the surgical positioning apparatus into group I and group II, wherein each group comprises three or more positioning markers;
(b) reading information on the positioning markers in group I and the group II in step (a) and information on the surgical positioning apparatus, and reading the image obtained by scanning in step (1);
(c) performing threshold-segmentation on the image obtained in step (b) and extracting and generating valid polygon data;
(d) fitting and deciding the polygon data obtained in step (c) according to the information on the surgical positioning apparatus obtained in step (b), thereby screening out the positioning markers in the image;
(e) calculating a distance between every two positioning markers of the positioning markers in the image obtained in step (d);
(f) choosing 3 positioning markers from the positioning markers on the surgical positioning apparatus in group I to constitute a triangle as a triangular template, and searching for a triangle in the image approximately identical to the triangular template; if there is no such triangle, choosing 3 positioning markers from the positioning markers on the surgical positioning apparatus in group II to constitute a triangle as a triangular template, and searching for a triangle in the image approximately identical to the triangular template; and if there is still no such triangle, choosing the positioning markers on the surgical positioning apparatus from group I and group II to constitute a triangle as a triangular template, searching for a triangle in the image approximately identical to the triangular template; and
(g) matching serial numbers of respective vertices of the paired congruent triangles according to a one-to-one correspondence, to form a matching vertex pair, and searching for an image positioning marker outside of the triangular template in the image corresponding to a positioning marker on the surgical positioning apparatus with reference to the congruent triangular template, until all image positioning markers match the positioning markers on the surgical positioning apparatus.

8. The surgical positioning system of claim 4, wherein the surgical robot is a robot arm with at least three translational degrees of freedom and three rotational degrees of freedom.

9. The surgical positioning system of claim 4, wherein a distance between any two of the reflecting balls is greater than 50 mm and a difference between various distances between the reflecting balls is greater than 5 mm; and at least three of the reflecting balls are at an angle less than or equal to 75°.

10. The surgical positioning system of claim 4, wherein the positioning markers are divided into two groups, wherein each group comprises three or more positioning markers, and the distribution of positioning markers in each group on the bracket satisfies the following conditions: a distance between any two positioning markers is greater than 20 mm and a difference between various distances between the positioning markers is greater than 5 mm; and at least three of the positioning markers are at an angle of less than or equal to 75°.

11. The surgical positioning system of claim 9, wherein the positioning markers are divided into two groups, wherein each group comprises three or more positioning markers, and the distribution of positioning markers in each group on the bracket satisfies the following conditions: a distance between any two positioning markers is greater than 20 mm and a difference between various distances between the positioning markers is greater than 5 mm; and at least three of the positioning markers are at an angle of less than or equal to 75°.

12. The surgical positioning system of claim 4, further comprising: a guiding device configured to be connected to the tip of the surgical robot.

13. The surgical positioning system of claim 4, wherein the optical tracking device is configured to identify the reflecting balls of the surgical positioning apparatus so as to track the surgical positioning apparatus.

14. The surgical positioning method of claim 6, wherein in step (3), the host computer calculates the transformation relation among the patient, the image and the surgical robot in a coordinate system in which the surgical positioning apparatus is located according to a rotation matrix and a translation vector between a coordinate vector of a robot tracer and a coordinate vector of the surgical positioning apparatus.

15. The surgical positioning method of claim 6, wherein step (3) further comprises: choosing one of a patient coordinate system, a robot coordinate system, a robot base coordinate system and an image coordinate system as the world coordinate system, and outputting a transformation relation by which the patient, the image and the surgical robot are brought into the common world coordinate system, as a result of image registration.

16. The surgical positioning method of claim 14, wherein step (3) further comprises: choosing one of a patient coordinate system, a robot coordinate system, a robot base coordinate system and an image coordinate system as the world coordinate system, and outputting a transformation relation by which the patient, the image and the surgical robot are brought into the common world coordinate system, as a result of image registration.

17. The surgical positioning method of claim 6, wherein a distance between any two of the reflecting balls is greater than 50 mm and a difference between various distances between the reflecting balls is greater than 5 mm; and at least three of the reflecting balls are at an angle less than or equal to 75°.

18. The surgical positioning method of claim 6, wherein the positioning markers are divided into two groups, wherein each group comprises three or more positioning markers, and the distribution of positioning markers in each group on the bracket satisfies the following conditions: a distance between any two positioning markers is greater than 20 mm and a difference between various distances between the positioning markers is greater than 5 mm; and at least three of the positioning markers are at an angle of less than or equal to 75°.

19. The surgical positioning method of claim 17, wherein the positioning markers are divided into two groups, wherein each group comprises three or more positioning markers, and the distribution of positioning markers in each group on the bracket satisfies the following conditions: a distance between any two positioning markers is greater than 20 mm and a difference between various distances between the positioning markers is greater than 5 mm; and at least three of the positioning markers are at an angle of less than or equal to 75°.

20. The surgical positioning method of claim 16 further comprising: according to a surgical route drawn in the image, expressing, with the host computer, spatial coordinates of the surgical route as a straight line in the world coordinate system; and controlling, with the host computer, the surgical robot to direct to the surgical route.

21. The surgical positioning method of claim 6 further comprising: monitoring, with the optical tracking device, a movement of the surgical positioning apparatus in real time; and calculating, with the host computer, a direction and magnitude of the movement, and controlling the surgical robot to correct its own motion according to the direction and magnitude of the movement.
